(19) United States
(12) Patent Application Publication
(10) Pub. No.: US 2003/0144347 A1
(43) Pub. Date: Jul. 31, 2003

(54) Method for treating autoimmune disease

(76) Inventor: Ralph Ryback, Bethesda, MD (US)

Correspondence Address: CONNOLLY BOVE LODGE & HUTZ LLP, SUITE 800, 1990 M STREET NW, WASHINGTON, DC, 20036-3425, US

(21) Appl. No.: 10/293,492

(22) Filed: Nov. 14, 2002

Related U.S. Application Data
(60) Provisional application No. 60/331,324, filed on Nov. 14, 2001.

(51) Int. Cl. A61K 31/35; A61K 31/36; A61K 31/255

(52) U.S. Cl. 514/459; 514/517; 514/464

(57) ABSTRACT

The present invention is directed to a method for treating autoimmune diseases comprising administering a therapeutically effective amount of a compound of the following formula (I)

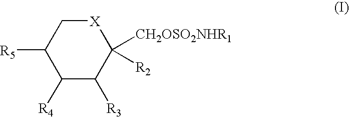

wherein X, R₁, R₂, R₃, R₄, R₅ and R₆ are as herein defined.

### FIELD OF THE INVENTION

The present invention is directed to a method for treating autoimmune diseases. According to the present invention, various sulfamate compounds, including topiramate, are used for treating autoimmune diseases.

### BACKGROUND OF INVENTION

Compounds of formula (1):

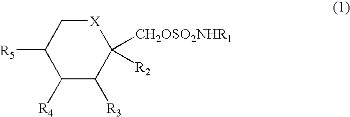

are known antiepileptic compounds that are highly effective anticonvulsants in animal tests (Maryannoff et al. J. Med Chem. 1987, 30, 880-887; Maryannoff et al., Bioorg. Med. Chem. Lett. 1993, 3, 2653-2656; Shank et al., Epilepsia 1994, 35, 450-460; Maryannoff et al., J. Med. Chem. 1998 41, 1315-1343). These compounds are described for example in U.S. Pat. No. 4,513,006, No. 5,242,942, and No. 5,384,327.

One of these compounds 2,3:4,5-bis-O-(1-methylethylidence)-β-D-fructopyranose sulfamate known as topiramate has been demonstrated in clinical trials of human epilepsy to be effective as adjunctive therapy or as monotherapy in treating simple and complex partial seizures and secondarily generalized seizures (Faught et al., Epilepsia 1995, 36 (Sr), 33; S. K. Sachdeo et al. Epilepsia 1995, 36 (S4), 33; GLAUSER, Epilepsia 1999, 40 (S5), S71-80; SACHDEO, Clin. Pharmacokiet 1998, 34, 335-346), and is currently marketed for the treatment of seizures in patients with simple and complex partial epilepsy and seizures in patients with primary or secondary generalized seizures in the United States, Europe and many other markets throughout the world.

Compounds of formula (1) were initially found to possess anticonvulsant activity in the traditional maximal electroshock seizure (MES) test in mice (Shank et al., Epilepsia 1994, 35, 450-460). Subsequent studies revealed that compounds of formula (1) were also highly effective in the MES test in rats. Topiramate was also found to effectively block seizures in several rodent models of epilepsy (Nakamura et al., Eur. J. Pharmacol. 1994, 254, 83-89), and in an animal model of kindled epilepsy (Wauquier et al., Epilepsy Res. 1996, 24, 73-77).

More recently, Shank et al., in U.S. Pat. No. 5,760,006 disclosed the use of compounds of formula (I) for the treatment of psoriasis. Shank et al., in PCT Publication WO 00/61138 disclose the use of compounds of formula (I) for the treatment of chronic neurodegenerative disorders. Turski, et al., in PCT Publication WO 00/01376 suggest the use of compounds of formula (I) along with a myriad of other compounds for the treatment of demyelinating disorders. Lomia in WO 00/66096 suggest the use of certain compounds of Formula (1) along with a number of other compounds for asthma.

Autoimmune diseases afflict millions of Americans. Most autoimmune diseases strike women more often than men; in particular, autoimmune diseases often affect women of working age and during their childbearing years.

The immune system is a complicated network of cells and cell components (called molecules) that normally work to defend the body and eliminate infections caused by bacteria, viruses, and other invading microbes. One of the classically accepted features of the immune system is the capacity of the immune system to distinguish between the self and the non-self or foreign materials within the body. Autoimmunity is the breakdown of one or more of the basic mechanisms regulating immune tolerance resulting in the immune system's inability to recognize the self as non-foreign. If a person has an autoimmune disease, the immune system mistakenly attacks the self, targeting the cells, tissues and organs of a person's own body. Autoimmune diseases are the result of the immune system's response to the self and the resulting pathologic consequences resulting from this self-reactivity. The essential feature of an autoimmune disease is therefore tissue injury caused by the immunologic reaction of the organism's immune system to its own tissues or other systems.

There are many different autoimmune diseases, and they can each affect the body in different ways. The different types of autoimmune diseases form a spectrum, from those specifically affecting a single organ to systemic disorders with involvement of many organs. Systemic autoimmune diseases differ from organ-specific diseases in that pathologic lesions for systemic autoimmune diseases are found in multiple, diverse organs and tissues. For example, in multiple sclerosis, the autoimmune reaction is directed against the brain, whereas in Crohn's disease, the autoimmune reaction is directed against the gut. In other autoimmune diseases such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus.

Most, if not all, autoimmune diseases can be classified as systemic because the disease is associated with the circulation of activated immune cells—immune cells that in turn target or manifest injury in specific tissues or organs. However, autoimmune diseases are often characterized as organ or tissue specific if the immune cells target (or localize at) specific organs or tissues and systemic if the immune cells target multiple organs or tissues.

Organ or tissue specific autoimmune disorders include, but are not limited to, Graves' disease, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, multiple sclerosis, Guillain-Barré syndrome, stiff-man syndrome, acute rheumatic fever, sympathetic ophthalmia, Goodpasture's syndrome, autoimmune uveitis, temporal artertis, Bechet's disease, Crohn's disease, ulcerative colitis, primary bilary cirrhosis, autoimmune hepatitis, autoimmune oophoritis, fibromayalgia, polymyositis, dermatomyostis, ankylosing spondylitis, psoriasis, Takayashu arteritis, panniculitis, pemphigoid, vasculitis of unknown origin, anca negative vasculitis and anca positive vasculitis.

Systemic specific autoimmune disorders include, but are not limited to, systemic lupus erythematosus, psoriatic arthritis, rheumatoid arthritis, schleroderma, systemic necrotizing vasculitis, Wegener's granulomatosis, CREST syndrome, antiphospholipid syndrome and Sjögren's syndrome.

In addition, there exist a number of disorders which exhibits possible autoimmune type expressions, such as eosinophilic gastroenteritis, atypical topical dermatitis, cardiomyopathy, postinfectious syndromes such as postinfectious endomyocarditis, and the like.

Autoimmune diseases may affect any organ or tissue systems including, but not limited to, the central or peripheral nervous system, the gastrointestinal system, the blood, endocrine glands, adrenal glands, skin, connective tissue, the skeletal system (including bones and joints), the respiratory system (including the lungs), the cardiovascular system (including blood vessels and the heart), genitalia, eyes, muscles, and the like.

Accordingly, significant efforts have been underway to develop treatments for autoimmune diseases. However, there still remains a need for methods for treating autoimmune diseases.

### SUMMARY OF INVENTION

It has been found according to the present invention that compounds of the formula (I), as defined below, are useful in treating autoimmune diseases. The present invention is directed to a method for treating an autoimmune disease, comprising administering to a subject in need thereof, a therapeutically effective amount of a compound of formula (I)

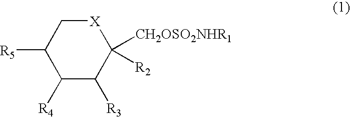

wherein

X is CH₂ or oxygen;

R₁ is hydrogen or alkyl and

R₂, R₃, R₄ and R₅ are independently hydrogen or lower alkyl and, when X is CH₂, R₄ and R₅ may be alkene groups joined to form a benzene ring and, when X is oxygen, R₂ and R₃ and/or R₄ and R₅ together may be a methylenedioxy group of the following formula (II):

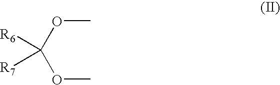

wherein

R₆ and R₇ are the same or different and are hydrogen, lower alkyl or are alkyl and are joined to form a cyclopentyl or cyclohexyl ring.

### DETAILED DESCRIPTION OF THE INVENTION

The present invention is directed to a method for treating autoimmune diseases, comprising administering to a subject in need thereof, a therapeutically effective amount of a compound of formula (I)

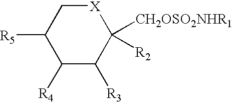

wherein X, R₁, R₂, R₃, R₄ and R₅ are as herein defined.

In an embodiment of the present invention is a method for treating an autoimmune disease comprising administering to a subject in need thereof a therapeutically effective amount of a compound of formula (I) wherein the compound of formula (I) is topiramate.

In an embodiment of the present invention, the therapeutically effective amount of the compound of formula (I) is in the range of about 5 to about 500 mg daily. In another embodiment of the present invention, the therapeutically effective amount of the compound of formula (I) is in the range of about 5 to about 250 mg daily. In a still further embodiment of the present invention, the therapeutically effective amount of the compound of formula (I) is in the range of about 5 to about 100 mg daily. In yet another embodiment of the present invention, the therapeutically effective amount of the compound of formula (I) is in the range of about 5 to about 50 mg daily. In yet another embodiment of the present invention, the therapeutically effective amount of the compound of formula (I) is in the range of less than about 50 mg daily.

In an embodiment of the present invention, the autoimmune disease is an organ or tissue specific autoimmune diseases. In another embodiment of the present invention, the autoimmune disease is a systemic autoimmune disease. In yet another embodiment of the present invention, the autoimmune disease is a disorder which exhibits autoimmune type expressions.

In yet another embodiment of the present invention, the autoimmune disease is autoimmune disease affecting a biological system selected from the group consisting of the central nervous system; the peripheral nervous system; the gastrointestinal system; the blood; blood vessels; the heart; an endocrine gland; an adrenal gland; the skin; the bones; the joints; the lungs; muscles; genitalia; eyes and connective tissue.

In an embodiment of the present invention is a method for treating an autoimmune disease selected from the group consisting of Graves' disease, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, Guillain-Barré syndrome, stiff-man syndrome, acute rheumatic fever, sympathetic ophthalmia, Goodpasture's syndrome, autoimmune uveitis, temporal artertis, Bechet's disease, Crohn's disease, ulcerative colitis, primary bilary cirrhosis, autoimmune hepatitis, autoimmune oophoritis, fibromayalgia, polymyositis, dermatomyostis, ankylosing spondylitis, Takayashu arteritis, panniculitis, pemphigoid, vasculitis of unknown origin, anca negative vasculitis, anca positive vasculitis, systemic lupus erythematosus, psoriatic arthritis, rheumatoid arthritis, schleroderma, systemic necrotizing vasculitis, Wegener's granulomatosis, CREST syndrome, antiphospholipid syndrome, Sjögren's syndrome, cosinophilic gastroenteritis, atypical topical dermatitis, cardiomyopathy, post-infectious syndromes and postinfectious endomyocarditis.

In another embodiment of the present invention, the autoimmune disease is selected from the group consisting of Graves' disease, Hashimoto's thyroiditis, autoimmune polyglandular syndrome, immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, Guillain-Barré syndrome, stiff-man syndrome, acute rheumatic fever, sympathetic ophthalmia, Goodpasture's syndrome, autoimmune uveitis, temporal artertis, Bechet's disease, Crohn's disease, ulcerative colitis, primary bilary cirrhosis, autoimmune hepatitis, autoimmune oophoritis, fibromayalgia, polymyositis, dermatomyostis, ankylosing spondylitis, Takayashu arteritis, panniculitis, pemphigoid, vasculitis of unknown origin, anca negative vasculitis, anca positive vasculitis, systemic lupus erythematosus, psoriatic arthritis, rheumatoid arthritis, schleroderma, systemic necrotizing vasculitis, Wegener's granulomatosis, CREST syndrome, antiphospholipid syndrome, Sjögren's syndrome, eosinophilic gastroenteritis, atypical topical dermatitis, cardiomyopathy, post-infectious syndromes and post-infectious endomyocarditis.

In another embodiment of the present invention, is a method for treating an autoimmune disease selected from the group consisting of Graves' disease, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, Guillain-Barré syndrome, stiff-man syndrome, acute rheumatic fever, sympathetic ophthalmia, Goodpasture's syndrome, autoimmune uveitis, temporal artertis, Bechet's disease, Crohn's disease, ulcerative colitis, primary bilary cirrhosis, autoimmune hepatitis, autoimmune oophoritis, fibromayalgia, polymyositis, dermatomyostis and ankylosing spondylitis, Takayashu arteritis, panniculitis, pemphigoid, vasculitis of unknown origin, anca negative vasculitis and anca positive vasculitis.

In another embodiment of the present invention, is a method for treating an autoimmune disease selected from the group consisting of systemic lupus erythematosus, psoriatic arthritis, rheumatoid arthritis, scleroderma, systemic necrotizing vasculitis, Wegener's granulomatosis, CREST syndrome, antiphospholipid syndrome and Sjögren's syndrome.

In another embodiment of the present invention, is a method for treating an autoimmune disease selected from the group consisting of eosinophilic gastroenteritis, atypical topical dermatitis, cardiomyopathy and postinfectious syndromes. In yet another embodiment of the present invention, is a method for treating an autoimmune disease selected from the group consisting of eosinophilic gastroenteritis, atypical topical dermatitis, cardiomyopathy, postinfectious syndromes and endomyocarditis.

In yet another embodiment of the present invention, the diseases treated according to the present invention include autoimmune diseases of the nervous system such as myasthenia gravis, autoimmune neuropathics such as Guillain-Barre and autoimmune uveitis; autoimmune diseases of the blood such as autoimmune hemolytic anemia, pernicious anemia and autoimmune thrombocytopenia; autoimmune diseases of the blood vessels such as temporal artertis; anti-phospholipid syndrome; vasculitides such as Wegener's granulomatosis and Behcet's disease; autoimmune diseases of the skin such as dermatitis herpetiformis, pemphigus vulgaris and vitiligo; autoimmune diseases of the gastrointestinal system such as Crohn's Disease, ulcerative colitis, primary bilary cirrhosis and autoimmune hepatitis; autoimmune diseases of the endocrine glands such as type 1 or immune-mediated diabetes mellitus, Grave's disease, Hashimoto's thyroiditis, autoimmune oophoritis and orchitis, autoimmune diseases of the adrenal gland; autoimmune diseases of the connective tissue disease such as rheumatoid arthritis, fibromyalgia, systemic lupus erythematosus, scleroderma, polymyositis, dermatomyostis; spondyloarthropathies such as ankylosing spondylitis and Sjogren's syndrome; and disorders exhibiting autoimmune type expressions such as eosinophilic gastroenteritis and atypical topical dermatitis.

In yet another embodiment of the present invention, the autoimmune disease is selected from the group consisting of psoriatic arthritis, rheumatoid arthritis, insulin dependent diabetes, insulin resistant diabetes, systemic lupus erythematosus, fibromalaygia, Grave's disease, Crohn's disease, pernicious amenia, temporal arteritis, ulcerative colitis, schleroderma and myasthenia gravis. Preferably, the autoimmune disease is selected from the group consisting of psoriatic arthritis, rheumatoid arthritis, systemic lupus erythematosus, fibromalaygia, Grave's disease, Crohn's disease, pernicious amenia, temporal arteritis, ulcerative colitis, scleroderma and myasthenia gravis.

In yet another embodiment of the present invention, the autoimmune disease is selected from the group consisting of pemphigus vulgaris, rheumatoid arthritis, psoriatic arthritis, ulcerative colitis and systemic lupus erythematosus.

In yet another embodiment of the present invention is a method for treating an immune related disease selected fron the group consisting of rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, nephrosis, atopic diseases, hypersensitity reactions, conjunctivitis, endometriosis, urticaria, systemic anaphalaxis, dermatitis, pernicious anemia, hemolytic disesease, thrombocytopenia, graft rejection of any organ or tissue, kidney translplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, myasthenia gravis, antibody-meditated cytotoxicity, type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, sclerodenma, mixed connective tissue disease, idiopathic Addison's disease, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (e.g., including but not limited to asthenia, anemia, cachexia, and the like), and chronic salicylate intoxication.

In yet another embodiment of the present invention is a method for the treatment of psoriasis comprising administering to a subject in need thereof less than about 50 mg daily of a compound of formula (I), preferably topiramate.

In yet another embodiment of the present invention is a method for the treatment of multiple sclerosis comprising administering to a subject in need thereof of less than about 50 mg daily of a compound of formula (I), preferably topiramate.

The sulfamate compound of the present invention are of the following general formula (I):

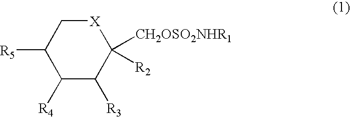

wherein

X is CH₂ or oxygen;

R₁ is hydrogen or alkyl and

R₂, R₃, R₄ and R₅ are independently hydrogen or lower alkyl and, when X is CH₂, R₄ and R₅ may be alkene groups joined to form a benzene ring and, when X is oxygen, R₂ and R₃ and/or R₄ and R₅ together may be a methylenedioxy group of the following formula (II):

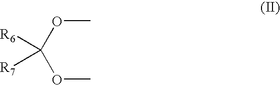

R₆ and R₇ are the same or different and are hydrogen, lower alkyl or are alkyl and are joined to form a cyclopentyl or cyclohexyl ring.

R₁ in particular is hydrogen or alkyl of about 1 to 4 carbons, such as methyl, ethyl and iso-propyl. Alkyl throughout this specification includes straight and branched chain alky. Alkyl groups for R₂, R₃, R₄, R₅, R₆ and R₇ are of about 1 to 3 carbons and include methyl, ethyl, iso-propyl and n-propyl. When X is CH₂, R₄ and R₅ may combine to form a benzene ring fused to the 6-membered X-containing ring, i.e., R₄ and R₅ are defined by the alkatrienyl group ═C—CH═CH—CH═.

A particular group of compounds of formula (I) is that wherein X is oxygen and both R₂ and R₃ and R₄ and R₅ together are methylenedioxy groups of the formula (II), wherein R₆ and R₇ are both alkyl such as methyl. A second group of compounds is that wherein X is CH₂ and R₄ and R₅ are joined to form a benzene ring. A third group of compounds of formula (I) is that wherein both R₂ and R₃ are hydrogen.

The compounds of formula (I) may be synthesized by the following methods:

(a) Reaction of an alcohol of the formula RCH₂OH with a chlorosulfamate of the formula CISO₂NH₂ or CISO₂NHR₁ in the presence of a base such as potassium t-butoxide or sodium hydride at a temperature of about −20 to 25 C. and in a solvent such as toluene, THF, or dimethylformamide wherein R is a moiety of the following formula (III):

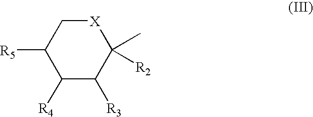

(b) Reaction of an alcohol of the formula RCH₂OH with a sulfurylchloride of the formula SO₂Cl₂ in the presence of a base such as triethylamine or pyridine at a temperature of about −40 to 25 C. in a solvent such as diethyl ether or methylene chloride to produce a chlorosulfate of the formula RCH₂OSO₂Cl may then be reacted with an amine of the formula R₁NH₂ at a temperature of about 40 to 25 C. in a solvent such as methylene chloride or acetonitrile to produce a compound of formula (I). The reaction conditions for (b) are also described by Tsuchiya, et al. in Tetrahedron Lett., 1978, 3365

(c) Reaction of the chlorosulfate RCH₂OSO₂Cl with a metal azide such as sodium azide in a solvent such as methylene chloride or acetonitrile yields an azidosulfate of the formula RCH₂OSO₂N₃ as described by Hedayatullah in Tetrahedron Lett. 1975, 2455. The azidosulfate is then reduced to a compound of formula (I) wherein R¹ is hydrogen by catalytic hydrogenation, e.g. with a noble metal and H₂ or by heating with copper metal in a solvent such as methanol.

The starting materials of the formula RCH₂OH may be obtained commercially or as known in the art. For example, starting materials of the formula RCH₂OH wherein both R₂ and R₃ and R₄ and R₅ are identical and are of the formula (II) may be obtained by the method of Brady in Carbohydr. Res. 1970, 14, 35 or by reaction of the trimethylsilyl enol ether of a R₆COR₇ ketone or aldehyde with fructose at a temperature of about 25° C., in a solvent such a halocarbon, e.g. methylene chloride in the presence of a protic acid such as hydrochloric acid or a Lewis Acid such as zinc chloride. The trimethylsilyl enol ether reaction is described by Larson, et al. in J. Org. Chem. 1973, 38 3935.

Further, carboxylic acids and aldehydes of the formulae RCOOH and RCHO may be reduced to compounds of the formula RCH₂OH by standard reduction techniques, e.g. reaction with lithium aluminum hydride, sodium borohydride or borane-THF complex in an inert solvent such as diglyme, THF or toluene at a temperature of about 0 to 100° C., e.g. as described by H. O. House in “Modern Synthetic Reactions”, 2^{nd} Ed., pages 45 to 144 (1972).

The compounds of formula (I) may also be made by the process disclosed U.S. Pat. No. 4,513,006, No. 5,242,942, No. 5,384,327 and No. 5,760,006 which are incorporated by reference herein.

The compounds of formula (I) include the various individual isomers as well as the racemates thereof, e.g., the various alpha and beta attachments, i.e., below and above the plane of the drawing, of R₂, R₃, R₄ and R₅ on the 6-membered ring. Preferably, the oxygen of the methylenedioxy group (II) is attached on the same side of the 6-membered ring.

As used herein, the term “autoimmune diseases” shall include organ or tissue specific autoimmune diseases, systemic autoimmune diseases and diseases which exhibit autoimmune type expression, more particualry, autoimmine diseases include Graves' disease, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, Guillain-Barré syndrome, stiff-man syndrome, acute rheumatic fever, sympathetic ophthalmia, Goodpasture's syndrome, autoimmune uveitis, temporal artertis, Bechet's disease, Crohn's disease, ulcerative colitis, primary bilary cirrhosis, autoimmune hepatitis, autoimmune oophoritis, fibromayalgia, polymyositis, dermatomyostis, ankylosing spondylitis, Takayashu arteritis, panniculitis, pemphigoid, vasculitis of unknown origin, anca negative vasculitis, anca positive vasculitis, systemic lupus erythematosus, psoriatic arthritis, rheumatoid arthritis, schleroderma, systemic necrotizing vasculitis, Wegener's granulomatosis, CREST syndrome, antiphospholipid syndrome, Sjögren's syndrome, eosinophilic gastroenteritis, atypical topical dermatitis, cardiomyopathy, post-infectious syndromes, postinfectious endomyocarditis.

Organ or tissue specific autoimmune diseases include, Graves' disease, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, Guillain-Barré syndrome, stiff-man syndrome, acute rheumatic fever, sympathetic ophthalmia, Goodpasture's syndrome, autoimmune uveitis, temporal artertis, Bechet's disease, Crohn's disease, ulcerative colitis, primary bilary cirrhosis, autoimmune hepatitis, autoimmune oophoritis, fibromayalgia, polymyositis, dermatomyostis and ankylosing spondylitis, Takayashu arteritis, panniculitis, pemphigoid, vasculitis of unknown origin, anca negative vasculitis and anca positive vasculitis.

Systemic autoimmune diseases include, systemic lupus erythematosus, psoriatic arthritis, rheumatoid arthritis, schleroderma, systemic necrotizing vasculitis, Wegener's granulomatosis, CREST syndrome, antiphospholipid syndrome and Sjögren's syndrome.

As used herein, the term “disorder which exhibit autoimmune type expressions” shall mean any disorder which results in elevated levels of auto-antibodies and is not otherwise characterized as an autoimmune disease. Suitable examples include, but are not limited to eosinophilic gastroenteritis, atypical topical dermatitis, cardiomyopathy, postinfectious syndromes such as postinfectious endomyocarditis, and the like.

As used herein, unless otherwise noted, the term “immune related diseases” shall mean any disease or disorder which is characterized by the presence of an immune system response that induces a cell, tissue, organ or multisystem pathology or which is due to a malfunction of any part of the immune systems, including rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, bums, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, nephrosis, atopic diseases, hypersensitity reactions, conjunctivitis, endometriosis, urticaria, systemic anaphalaxis, dermatitis, pernicious anemia, hemolytic disesease, thrombocytopenia, graft rejection of any organ or tissue, kidney translplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, myasthenia gravis, antibody-meditated cytotoxicity, type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (e.g., including but not limited toasthenia, anemia, cachexia, and the like), chronic salicylate intoxication.

As used herein, unless otherwise noted, the term “subject” or “patient” shall mean an animal, preferably a mammal, more preferably a human, who is the object of treatment, observation or experiment.

As used herein, unless otherwise noted, the term “therapeutically effective amount” shall mean that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The following non-limiting examples are presented to further illustrate the present invention and to aid in the understanding of the invention, and are not intended and should not be construed to limit in any way the invention set forth in the claims which follow hereafter.

### EXAMPLE 1

A patient suffering from multiple sclerosis was administered 15 mg per day of topiramate. The dosage was increased after 15 days to 30 mg per day. At 15 mg, the patient's involuntary stretch rigid response stopped and the patient was able to get out of bed without falling backward when his feet touched the floor for the first time in nine years. However, the higher dose of 30 mg reversed this benefit and the dose was then lowered to 12.5 mg per day. This spastic rigid response is no longer present and the beneficial effect has continued for more than 10 months.

### EXAMPLE 2

A patient suffering from diabetes, Type 1, was administered 25 mg per day of topiramate for 10 days, which was then increased to 50 mg per day. The patient's blood glucose levels began to decrease with no change in diet or insulin dose from peaks of 375 to 400 mg/DL and averaging above 220 to a range of 180 to 220 mg/DL after a three week period with a further reduction to between 160 and 180 mg/DL after three more weeks and remaining in that range for a follow-up period of 16 weeks.

### EXAMPLE 3

A patient suffering from rheumatoid arthritis was administered 25 mg per day of topiramate with an increase every 10 days by 25 mg until 25 mg, 3 times per day, was achieved. The patient previously took 3 or 4 tablets per day of lortab, seven 2.5 mg tablets of methotrexate once a week, 200 mg per day of minocycline and 10 mg per day of prednisone.

Upon the treatment with topiramate, the patient reported a significant decrease in pain and was able to slowly discontinue the use of lortab (i.e. hydrocodone bitartrate), a semisynthetic narcotic analgesic, without the reoccurrence of pain. Also, the prednisone was reduced to 5 mg per day, the minocycline was discontinued, and the methotrexate was decreased to five 2.5 mg tablets once a week, with decreased pain and increased mobility. This benefit has continued for 15 weeks.

### EXAMPLE 4

An adolescent patient suffering from rheumatoid arthritis began taking 15 mg per day of topiramate for one week, which was then increased by 15 mg weekly until 45 mg per day, in three divided dosages of 15 mg each, was reached. The patient previously took 3 to 5 mg of prednisone daily. At 15 mg twice a day, the patient described a 40-50% decrease in pain, which increased to a 70% reduction at 45 mg per day. Over the next two months, the prednisone was decreased to 0.5 mg/day with maintenance of benefits. This has continued for four months.

### EXAMPLE 5

A patient suffering from psoriatic arthritis began taking 15 mg per day of topiramate. After one week the dose was increased to 15 mg twice per day, at which time there was some improvement in skin lesions and decreased joint pain. After a further increase to 15 mg every morning and 30 mg every evening, the patient was able to more easily get out of bed after one month at this dose. When the dose was increased slightly to 25 mg twice a day, the patient realized he was experiencing minimal pain and resumed garden work using a rototiller for the first time in two years. These benefits have continued for more than five months at this dose.

### EXAMPLE 6

A patient suffering from psoriatic arthritis began taking 25 mg per day of topiramate, but the patient wanted to stop medication after 4 weeks in spite of a significant reduction and almost total disappearance of her middle knuckle swelling, because of severe lethargy. The dose was reduced to 7.5 mg twice a day and the improvement continued in spite of winter weather. This has continued for more than ten months.

### EXAMPLE 7

A patient suffering from psoriatic arthritis began taking 25 mg per day of topiramate. The dosage was increased each week until 45 mg per day was achieved. The patient reported a significant decrease in pain and greater mobility, which persisted even when stressed. The patient was stabilized at 25 mg, twice a day, for convenience. His benefits from topiramate have continued for almost five months.

### EXAMPLE 8

A patient suffering from ulcerative colitis began taking 7.5 mg per day of topiramate, resulting in a decrease in mucous after 7 days and no mucous or bleeding (i.e. light stools) after two weeks. When the dose was increased to 15 mg per day, the patient complained about being tired during the day. After two weeks at this dose, the patient's stools became darker with increase bowel frequency of movements and mucous. The patient stopped the topiramate for six days and then resumed it at 7.5 mg per day with a decrease in mucous, bowel movements and bleeding, which has persisted for more than 16 weeks.

### EXAMPLE 9

A patient suffering from ulcerative colitis began taking 25 mg per day of topiramate, with dosage increases of 25 mg/day each week. At a dose of 50 mg twice a day, the patient noted a decreased intensity of symptoms, and at 75 mg twice a day reported decreased frequency of symptoms. At 100 mg twice a day, the patient reported that his diarrhea had stopped and that he was having normal daily bowel movements in spite of having reduced asakol dosage, first from 1200 to 800 mg, and then to 400 mg twice a day. By the end of nine months, the patient's weight was decreased to 270 lbs from 315 lbs. His disease was no longer affecting his ability to go to or stay at work. His colonoscopy exam was negative for the first time since the onset of his illness.

### EXAMPLE 10

A patient suffering from ulcerative colitis began taking 15 mg per day of topiramate, which resulted in a significant decrease in bowel frequency, bleeding and mucous within one week. When the dose was increased to 30 and then to 45 mg per day, there was a reversal of benefits and then a further exacerbation of systems, with increased bowel frequency, bleeding and mucous above the base line while the patient was on prednisone and colozol. The topiramate was discontinued for three weeks and then resumed at a dose of 7.5 mg per day, with a significant decrease in frequency of bowel movement, bleeding and mucous, which has persisted for 18 weeks.

### EXAMPLE 11

A young adolescent patient suffering from systemic lupus erythematosis and taking 30 mg bid of prednisone began taking 15 mg per day of topiramate. After seven days with minimal change, the dose was increased to 15 mg twice a day, at which time a pre-existing generalized facial and body rash the patient had began to subside, headaches disappeared and joint pain greatly diminished. In addition, the patient's carbohydrate craving, especially for sweets, was markedly reduced with a striking improvement in energy and mood over the next two weeks and concomitantly, the patient's antinuclear antibodies ANA was 2+ after 3 weeks of topiramate which had been 4+. The topiramate was increased to 15 mg in the morning and 30 mg every evening with further improvement. The prednisone was reduced to 25 mg per day after 4 weeks and to 10 mg after 6 weeks. This benefit has continued for over 6 months.

### EXAMPLE 12

A patient suffering from psoriasis began taking 15 mg per day of topiramate for seven days, which was increased by 15 mg per day until reaching a dose of 45 mg/day in equally divided doses. After 10 days at 45 mg, the patient's lesions began to decrease with a decrease in the psoriasis area and severity index (PASI) on 25 mg bid given for convenience from 12.2 to 5.4 which occurred within 8 weeks and was maintained at 16 weeks.

### EXAMPLE 13

A patient suffering from pemphigus vulgaris and taking 15 mg of chobetesol propionate per day began taking 15 mg per day of topiramate. The dose was increased to 45 mg in divided dosages over a three week period. The patient's lesions began to shrink, dried up and formed a scab, which came off leaving normal skin. This occurred and was maintained even after chobetesol propionate was reduced to 5 mg every 3 days, at 8 weeks from the initial dose of 15 mg per day. This improvement continued to be maintained at 12 weeks.

### EXAMPLE 14

A patient suffering from permphigus vulgaris and taking 50 mg twice a day of immuran and 20 mg per day of prednisone began taking 15 mg per day of topiramate. The dose was increased to 45 mg per day in divided dosages over a three-week period. At this time, one lesion was shrinking, and by four weeks it was healed and the second lesion was beginning to shrink. This occurred in spite of reducing the immuran to 50 mg and prednisone to 10 mg each day, which were their lowest dosages in two years. The dosage was raised to 30 mg twice a day with the second lesion healing. These improvements continued to be maintained at 20 weeks and Ig antibody was zero.

### EXAMPLE 15

A patient suffering from multiple autoimmune diseases was treated with topiramate. The patient was suffering from systemic lupus erythematosis (SLE) acquired anemia of gamma globulins, rheumatoid arthritis, Renaud's phenomena and Hashimotos thyroiditis. The patient was also taking medrol at doses of 32 mg per day and up to 132 mg per day and 25 mg weekly injections of methotrexate.

The topiramate treatment was begun at 15 mg per day and increased weekly by 15 mg to 65 mg per day and then stabilized at 50 mg per day after two months. The patient noted a very significant decrease in pain when walking and began to reduce the dose of medrol by 1 mg per week to a new maintenance dose of 5 mg per day. The frequency and intensity of her iritis, which was occurring every one to two months and lasting for 1 to 2 weeks, described as an “incredible pain relieved only by wrapping my eyes and head with a black towel”, decreased. It has occurred only once since using Topamax® when her father was dying. More revealing is the medication dosages and their frequency for her iritis (i.e. pred forte and cloxan drops) have been reduced remarkably and the patient has not had another episode in five months. All this improvement continued despite the cessation of methotrexate. The patient drove to her last appointment, which is the first time she has been able to do so in five years. She has now had consistent benefits for more than 6 months.

### EXAMPLE 16

A patient suffering from psoriasis began taking 15 mg per day of topiramate for 7 days with some improvement. When the dose was increased to 30 mg/day in divided dosages, the lesion began to get worse and at 45 mg/day in divided doses there was a clear deterioration. A 60 mg per day, new lesions were occurring. The dose was decreased to 15 mg every other day with minimal improvement. Within the first eight weeks, the PASI was judged to be unchanged at 29.1.

### EXAMPLE 17

A patient suffering from rheumatoid arthritis with a positive rheumatic factor about 3 decades earlier, at which time he also experienced polyarthritis and Renaud's phenomena, was treated with prednisone 40 mg per day for six months and was then weaned off the prednisone after one year. In the ensuing years, psoriasis developed on both elbows which was worse in the winter. He was an avid golfer and experienced painful swelling and stiffness of both wrists and hands, especially the left, and back pain and stiffness. Naproxen proved ineffective and he refused to consider a trial of steroids. A repeat rheumatoid factor was positive. He was begun on Topamax 15 mg a day which was increased weekly by 15 mg until 45 mg per day was taken in divided doses. His rheumatoid pain on a self-rating scale of 0 to 10 with ten being very severe decreased as follows: joint pain in both hands 6 to 2, wrist pain 7 to 2 or 3, back pain and stiffness 6 to 8 to 2 or 3. He reported he now sleeps better and “when I get up, I'm not stiff”. This benefit has continued for six months along with improvement in his psoriasis.

### EXAMPLE 18

A patient suffering from multiple sclerosis (M.S.) was treated with Topamax 7.5 mg at bedtime for three days during which time she was observed to be walking better and reported reduced back pain but continued cramping in both legs. The patient previously took trazadone 100 mg at bedtime, clonazepan 1 mg three times a day, citalapram 40 mg each morning, gabapentrin 600 mg four times a day, and synthroid 25 mcg per day for two years. She had refused steroids and interferon because of side effects.

The Topamax was increased to 15 mg at bedtime and she reported “no bad bladder accidents” and that her leg and foot cramps had disappeared. Two days later she was observed walking without a cane “straighter than I have in 10 years” and she reporting no urinary incontinence or back pain. She performed perfectly on the process noted cognitive functioning and felt her thought process was greatly improved. She was discharged on Topamax 15 mg at bedtime along with previous medications. The patient was much improved medically and emotionally.

The patient returned four weeks later walking with a cane, having worked “10 to 14 hours per day and exhausted from my move with little sleep during the past month”. She reported “insatiable itching all over my body wherever my clothes touch the skin” which she unsuccessfully tried to treat with cetirizine. She said she felt “dizzy, weak, Charlie horses in arches of feet and toes, unable to stand for more than 10 minutes before starting to have gait problems, problems sleeping, having trouble mentally and urine leaking”. Topamax was increased to 30 mg at bedtime and she was seen eight days later, at which time she reported “no dizziness, weakness, urinary incontinence and decreased gait problems, itching and pain.” She noted she was “sleeping wonderfully” and this benefit has continued for the past four months or until the present time. Her working diagnoses are Axis I Bipolar disorder, not otherwise specified Axis III multiple sclerosis.

### EXAMPLE 19

A patient presented with rapid mood swings and a complaint of severe intense itching from clothing tags and some clothing, which began over twenty years ago. She was begun on gabapenten which was increased to 300 mg qid. She returned relating that she felt a little calmer but complained that she had gained five pounds in two weeks. Her itching was unchanged. She was begun on Topamax 12.5 mg at bedtime which was increased weekly by 12.5 mg until 50 mg. She reported “true stabilization of my mood” and for the first time she noted no itching at all.

### EXAMPLE 20

A patient presented depressed related to a 15 pound weight gain following a recurrent of eosinophilic gastroenteritis treated with 20 mg of prednisone. This was his third bout in two years, all confirmed by a stomach biopsy. Each recurrence began between four and five months after being withdrawn from steroids, which had caused physical side effects and suspiciousness. A trial of Topamax 7.5 mg every three days and then 7.5 mg every other day was begun. The patient was again slowly withdrawn from prednisone and has not had a recurrence of his symptoms in 19 months.

The compounds of the present invention when employed to treat psoriasis or multiple sclerosis are typically administered in daily dosages of less than 50 mg, more typically at least about 5 mg., preferably about 5 to about 45 mg. and more preferably about 30 to about 45 mg when treating psoriasis. Dosages above 50 mg when treating psoriasis (e.g. 100 mg/day) resulted in worsening of lesions. When treating multiple sclerosis, the more preferred dosage is about 5 to about 30 mg. It has been observed that dosages above 30 mg. have resulted in worsening of the disease.

When used to treat autoimmune diseases other than psoriasis or multiple sclerosis, the daily dosage is typically up to about 100 mg., but in some cases could be up to about 250 mg. or even up to about 500 mg., more typically about 50 mg or less, and preferably about 5 to about 45 mg. A typical example is about 7.5 mg.

Optimal dosages and dosage regimens to be administered may be readily determined by those skilled in the art, and will vary with the pharmacodynamics characteristics of the particular agent, its time and mode of administration, the strength of the preparation and the advancement of the disease condition (including the nature and extent of the symptoms of the disease). In addition, factors associated with the particular patient being treated, including patient's sex, age, weight, diet, physical activity and concomitant diseases, will result in the need to adjust dosages and/or regimens.

For pharmaceutical administration, one or more of compound(s) of formula (I) may be administered by any suitable means, as would be apparent to one skilled in the art. More particularly, the compound(s) of formula (I) may be administered by any parenteral method, including, but not limited to oral, pulmonary, intraperitoneal (ip), intravenous (iv), intramuscular (im), subcutaneous (sc), transdermal, buccal, nasal, sublingual, ocular, rectal and vaginal. The compounds of formula (I) may further be administered topically, by any suitable method known to one skilled in the art, for example as a lotion, and the like. It will be readily apparent to those skilled in the art that any dose or frequency of administration that provides the therapeutic effect described herein is suitable for use in the present invention.

The compounds of formula (I) may be administered via a pharmaceutical composition comprising the compound of formula (I) and a pharmaceutically acceptable carrier.

Pharmaceutically acceptable carriers include, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds of this invention can be administered by any conventional method available for use in conjunction with pharmaceuticals, either as individual therapeutic agents or in a combination of therapeutic agents.

Dosage forms (compositions suitable for administration) typically contain about 1 mg to about 50 mg of active ingredient per unit. In these pharmaceutical compositions, the active ingredient will ordinarily be present in an amount of about 0.5-95% weight based on the total weight of the composition.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism, lotion or ointment.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parental administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethlcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alklypyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophilelipophile formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J. B. Lippincott Co., Philadelphia, Pa.

Moreover, the compounds of the present invention can be administered in the form of nose drops, or metered dose and a nasal or buccal inhaler. The drug is delivered from a nasal solution as a fine mist or from a powder as an aerosol.

The foregoing description of the invention illustrates and describes the present invention. Additionally, the disclosure shows and describes representative preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

What is claimed is:

1. A method for treating an autoimmune disease in a subject in need thereof comprising administering a therapeutically effective amount of a compound of the formula (I): 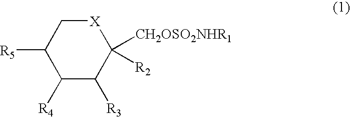
X is CH₂ or oxygen;
R₁ is hydrogen or alkyl and
R₂, R₃, R₄ and R₅ are independently hydrogen or lower alkyl and, when X is CH₂, R₄ and R₅ may be alkene groups joined to form a benzene ring and, when X is oxygen, R₂ and R₃ and/or R₄ and R₅ together may be a methylenedioxy group of the following formula (II): 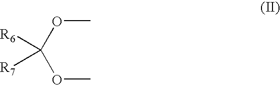
wherein
R₆ and R₇ are the same or different and are hydrogen, lower alkyl or are alkyl and are joined to form a cyclopentyl or cyclohexyl ring.

2. The method of claim 1, wherein the compound of formula (I) is topiramate.

3. The method of claim 1, wherein the therapeutically effective amount of the compound of formula (I) is in the range of about 5 to about 500 mg daily.

4. The method of claim 3, wherein the therapeutically effective amount of the compound of formula (I) is in the range of about 5 to about 250 mg daily.

5. The method of claim 4, wherein the therapeutically effective amount of the compound of formula (I) is in the range of about 5 to less than 50 mg daily.

6. The method of claim 1, wherein the autoimmune disease is selected from the group consisting of an organ or tissue specific autoimmune disease, a systemic autoimmune disease and a disorder exhibiting autoimmune expressions.

7. The method of claim 1, wherein the autoimmune disease is a disease affecting a biological system selected from the group consisting of the central nervous system; the peripheral nervous system, the gastrointestinal system; the blood; the blood vessels; the heart; an endocrine gland; an adrenal gland, connective tissue; bones; joints; lungs; muscles; genitalia; eyes and skin.

8. The method of claim 1, wherein the autoimmune disease is selected from the group consisting of Graves' disease, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, Guillain-Barré syndrome, stiff-man syndrome, acute rheumatic fever, sympathetic ophthalmia, Goodpasture's syndrome, autoimmune uveitis, temporal artertis, Bechet's disease, Crohn's disease, ulcerative colitis, primary bilary cirrhosis, autoimmune hepatitis, autoimmune oophoritis, fibromayalgia, polymyositis, dermatomyostis, ankylosing spondylitis, Takayashu arteritis, panniculitis, pemphigoid, vasculitis of unknown origin, anca negative vasculitis, anca positive vasculitis, systemic lupus erythematosus, psoriatic arthritis, rheumatoid arthritis, schleroderma, systemic necrotizing vasculitis, Wegener's granulomatosis, CREST syndrome, antiphospholipid syndrome, Sjögren's syndrome, eosinophilic gastroenteritis, atypical topical dermatitis, cardiomyopathy, post-infectious syndrome and postinfectious endomyocarditis.

9. The method of claim 6, wherein the autoimmune disease is an organ or tissue specific autoimmune disease.

10. The method of claim 9, wherein the autoimmune disease is selected from the group consisting of Graves' disease, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, Guillain-Barré syndrome, stiff-man syndrome, acute rheumatic fever, sympathetic ophthalmia, Goodpasture's syndrome, autoimmune uveitis, temporal artertis, Bechet's disease, Crohn's disease, ulcerative colitis, primary bilary cirrhosis, autoimmune hepatitis, autoimmune oophoritis, fibromayalgia, polymyositis, dermatomyostis, ankylosing spondylitis, Takayashu arteritis, panniculitis, pemphigoid, vasculitis of unknown origin, anca negative vasculitis and anca positive vasculitis.

11. The method of claim 6, wherein the autoimmune disease is a systemic autoimmune disease.

12. The method of claim 10, wherein the autoimmune disease is selected from the group consisting of systemic lupus erythematosus, psoriatic arthritis, rheumatoid arthritis, schleroderma, systemic necrotizing vasculitis, Wegener's granulomatosis, CREST syndrome, antiphospholipid syndrome and Sjögren's syndrome.

13. The method of claim 6, wherein the autoimmune disease is a disorder exhibiting autoimmune expressions.

14. The method of claim 13, wherein the autoimmune disease is selected from the group consisting of eosinophilic gastroenteritis, atypical topical dermatitis, cardiomyopathy, postinfectious syndromes and endomyocarditis.

15. The method of claim 6, wherein the autoimmune disease is selected from the group consisting of myasthenia gravis, autoimmune uveitis, autoimmune hemolytic anemia, pernicious anemia, autoimmune thrombocytopenia; temporal artertis; antiphospholipid syndrome, Behcet's disease; dermatitis herpetiformis, pemphigus vulgaris, vitiligo; Crohn's Disease, ulcerative colitis, primary bilary cirrhosis, autoimmune hepatits; type 1 or immune-mediated diabetes mellitus, Grave's disease, Hashimoto's thyroiditis, autoimmune oophoritis, orchitis, rheumatoid arthritis, fibromyalgia, systemic lupus erythematosus, scleroderma, polymyositis, dermatomyostisand Sjogren's syndrome.

16. The method of claim 6, wherein the autoimmune disease is selected from the group consisting of psoriatic arthritis, rheumatoid arthritis, insulin dependent diabetes, insulin resistant diabetes, systemic lupus erythematosus, fibromalaygia, Grave's disease, Crohn's disease, pernicious amenia, temporal arteritis, ulcerative colitis, schleroderma and myasthenia gravis.

17. The method of claim 6, wherein the autoimmune disease is selected from the group consisting of pemphegus vulgaris, rheumatoid arthritis, psoriatic arthritis, ulcerative colitis and systemic lupus erythematosus.

18. A method for treating an immune related disease in a subject in need thereof comprising administering a therapeutically effective amount of a compound of the formula (I): 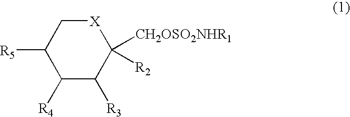
X is CH₂ or oxygen;
R₁ is hydrogen or alkyl and
R₂, R₃, R₄ and R₅ are independently hydrogen or lower alkyl and, when X is CH₂, R₄ and R₅ may be alkene groups joined to form a benzene ring and, when X is oxygen, R₂ and R₃ and/or R₄ and R₅ together may be a methylenedioxy group of the following formula (II): 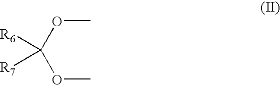
wherein
R₆ and R₇ are the same or different and are hydrogen, lower alkyl or are alkyl and are joined to form a cyclopentyl or cyclohexyl ring.

19. The method of claim 18, wherein the compound of formula (I) is topiramate.

20. The method of claim 18, wherein the immune related disease is selected from the group consisting of rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, bums, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, nephrosis, atopic diseases, hypersensitity reactions, conjunctivitis, endometriosis, urticaria, systemic anaphalaxis, dermatitis, pernicious anemia, hemolytic disesease, thrombocytopenia, graft rejection of any organ or tissue, kidney translplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, myasthenia gravis, antibody-meditated cytotoxicity, type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (including asthenia, anemia, cachexia) and chronic salicylate intoxication.

21. A method for the treatment of psoriasis or multiple sclerosis comprising administering to a subject in need thereof of less than about 50 mg daily of a compound of formula (I) 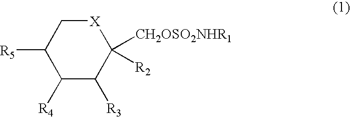
X is CH₂ or oxygen;
R₁ is hydrogen or alkyl and
R₂, R₃, R₄ and R₅ are independently hydrogen or lower alkyl and, when X is CH₂, R₄ and R₅ may be alkene groups joined to form a benzene ring and, when X is oxygen, R₂ and R₃ and/or R₄ and R₅ together may be a methylenedioxy group of the following formula (II): 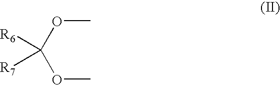
wherein
R₆ and R₇ are the same or different and are hydrogen, lower alkyl or are alkyl and are joined to form a cyclopentyl or cyclohexyl ring.

22. The method as in claim 21, wherein the compound of formula (I) is topiramate.
